(19) United States
(12) Patent Application Publication
(10) Pub. No.: US 2025/0001029 A1
(43) Pub. Date: Jan. 2, 2025

(54) SANITISING SYSTEMS AND METHODS

(71) Applicant: Ecochem Australia Pty Ltd, Upper Plenty, Victoria (AU)

(72) Inventors: Geoffrey Grant Literski, Upper Plenty, Victoria (AU); David Campbell, Gowanbrae, Victoria (AU)

(21) Appl. No.: 18/562,286

(22) PCT Filed: Jan. 4, 2022
(86) PCT No.: PCT/AU2022/050002

§ 371 Date: Nov. 17, 2023

Related U.S. Application Data
(60) Provisional application No. 63/189,742, filed on May 18, 2021.

(51) Int. Cl. A61L 2/22; A01N 25/06; A01N 37/02; A01P 1/00; A61L 2/18; A61L 2/26; A61L 11/00; A61L 101/36; B65B 55/02

(52) U.S. Cl. CPC A61L 2/22; A01N 25/06; A01N 37/02; A01P 1/00; A61L 2/18; A61L 2/26; A61L 11/00; B65B 55/02; A61L 2101/36; A61L 2202/14; A61L 2202/15; B65B 2210/06

(57) ABSTRACT

The present invention is directed to sanitising methods and systems. The methods and systems include sanitising surfaces in a food processing or food packaging facility, sanitising recyclable material, and sanitising agricultural material, among other materials. The methods include the steps of providing one or more sanitising liquid delivery lines for receiving a sanitising liquid; connecting the one or more delivery lines to one or more liquid spraying devices adapted to apply the sanitising liquid; wherein the sanitising liquid includes a composition including decanoic acid in a range between about 1% and about 5% w/w; and the sanitising liquid composition is diluted with a diluent to a concentration in a specified range. The methods also describe the type of spraying devices that are suitable in a range of applications. The systems of the invention include the components set out in the methods of the invention.

### RELATED APPLICATIONS

The present invention claims priority to U.S. provisional patent application No. 63/189,742 entitled “Sanitizing Systems and Methods” filed 18 May 2021, and to Australian patent application no. 2021221845 filed 25 Aug. 2021, the entire content of each of which is incorporated herein by reference.

### TECHNICAL FIELD

The present invention relates to sanitising or cleaning systems and methods, and more particularly the invention relates to systems and methods for sanitising material, including aggregate material, agricultural produce, food producing trees and hard surfaces.

### BACKGROUND

The invention has been developed primarily for use in, and/or with, the cleaning or sanitising of recycled glass cullet, however it will be appreciated that the invention may be applied in the cleaning or sanitising of other materials such as non-crushed glass plastics, steel, aluminum, or other hard materials, including combinations of such materials, and particularly recyclable materials such as those used in building and construction, including road construction. The invention has also been developed for use in the cleaning of hard surfaces such as the surfaces of machinery, processing or manufacturing equipment, other equipment or other hard surfaces.

The invention is suitable for cleaning and/or sanitising organic material, or partly organic material, such as soils, as well as aggregate material such as rocks. The invention is also suitable for sanitising agricultural produce, to extend the lifespan of the produce.

The invention provides systems and methods for sanitising plants and food producing trees. In particular it is suitable for sanitising citrus trees to eliminate and/or inhibit growth of fungi on leaves, twigs and the trunk of trees.

The invention is able to be used to sanitise the surfaces of food transportation equipment or food transportation vehicles, such as food trays or other large food conveying receptacles, or feed trucks.

In many applications, the process of sanitising or cleaning has a principal function of odour reduction or management of the material.

The invention is also able to be used for suppressing dust or other particles, together with the sanitising application.

The invention may also be used for cleaning or sanitising new products and packaging, such as metal, glass or plastic containers, whether in recyclable or non-recyclable form. Additionally the invention is able to be used in food preparation areas to reduce or eliminate bacteria such as listeria present on surfaces to which food or food packaging is exposed. This is particularly beneficial where such bacteria are commonly resistant to other sanitising methods.

The systems and methods of the invention may be applied in many settings, but particularly settings where cleaning, sterilising, decontaminating, sanitising or the control of pathogens is desired in industrial, commercial and other facilities, and particularly where the cleaning or sanitising of material, machinery, equipment, apparatus or the working environment more generally is important.

The process of re-using, re-purposing or recycling material, including by finding secondary or higher order uses for waste material, rarely, if ever, provides 100% reusability over the long term, even where the most innovative and costly methods are applied. There remain forces working against the widespread adoption of recycling practices, such as where it may be substantially more costly to recycle waste material into a product than to produce that product from the raw materials (even when resource consumption and waste management taxes are levied, for example, on single use products), or where there is just not sufficient technology to enable the recycling, at any cost. Even where efficient recycling processes are adopted, there is increasing demand for improvements to those processes, such as by increasing the amount of material that can be recycled (i.e. using large scale recycling machinery) to obtain scale-related benefits, or making processes more streamlined.

Recyclable materials, in many applications, need to be sanitised or cleaned of contamination. This is especially true where the intended use, or reuse, of the material is in applications which do not require it to be heated to very high temperatures (e.g. over 100 or 150 degrees) during the preparation or manufacture of a product including the recyclable material. For example, where recyclable components are used in aggregate, concrete, cement, asphalt or other road or hard-wearing surfaces, or pipe-laying, the recyclable material component may not be heated to elevated temperatures.

Further, it can be difficult and costly to prepare some materials for reuse due to the uniformity of condition required for some materials to be recycled in certain applications. For example, it can be costly to sort glass cullet by color, yet for many applications it is required that the glass cullet be substantially a uniform color to be able to be recycled.

Additionally, where a material is exposed to pathogens or other contaminants, it can be hard to completely remove these from the material. This is partly because some pathogens (e.g. bacteria) can have very long life-spans and survive, even under harsh conditions, until conditions are more suitable for growth of the pathogen. Furthermore, many sanitising products do not actively operate over the long term, for many reasons such as that they wash off surfaces when exposed to rain, or that they are not adapted to sanitise where material is re-infected due to animal excrement, such as bird or rodent droppings.

Further, where material is stored in open air conditions and exposed to elevated or low temperatures, wind and rain, effective sanitising may be more difficult.

Also, applying broad spectrum sanitising products, or numerous types of sanitising products to achieve the same result, can be very costly. Furthermore, simple cleaning agents such as detergents do not work well against pathogens, and more complex and costs agents may be required. This means that, where a sanitising agent is used, it can be important to conserve the volume of the agent for the cleaning process to be cost effective.

In the past, cleaning of materials during the stages preceding the re-use or re-purposing of those materials, has been performed by running the material through a cleaning bay filled with a detergent. Such bays have included means to vibrate and/or shake the material such that it is more effectively cleaned. Alternative cleaning systems for recyclable materials include large scale cleaning machines that function by enclosing, or substantially enclosing, the material in a cavity where a mixture of water and detergents is applied at increased temperatures such as around 80 or 90 degrees.

Previous cleaning or sanitising systems have not effectively removed certain contaminants, and particular pathogens, from recyclable material. This is partly due to traditional cleaning agents not effectively operating over the long term. Further, previous recycling operations have not been able to cost-effectively sort materials with sufficient uniformity for reuse.

Additionally, material, particularly recyclable material, that is processed in the manner described above and stored out of doors or in otherwise exposed or accessible spaces, may be subject to attracting wildlife (e.g. birds or rodents) that are attracted to the pathogens, and more particularly the smell of the certain bacteria. Since the material may be contaminated or damaged due to exposure to wildlife, maintaining cleanliness and sanitisation of the material is of utmost importance. Also, the health of those persons managing and working on site may be impacted directly by the presence of pathogens on the work-site (e.g. due to viral or bacterial spread). Further, some workers may be less willing to work on sites where bad odour resulting from pathogen growth on material or surfaces is unpleasant or offensive. In some contexts, the increased presence of wildlife and/or their droppings, due to wildlife being attracted to the odour producing pathogens, can also have a negative operational impact on work-sites.

There is a need for improved sanitising and cleaning systems and methods in the context of recycling sites such as transfer stations, resource recovery centres as set out above, and also in other areas where dust, odour, pests and contamination make the management of material difficult, or negatively impact working conditions, from an occupational health and safety perspective or otherwise.

Many of the same issues discussed above confronting the cleaning of recyclables apply to the cleaning of material to be used in the manufacture of products that are made from non-recyclables. For example, in the manufacturing of food storage containers, where removing contaminants from, or minimising the contamination of, material in those products is an important part the manufacturing process. Where contamination is a source of malodours, this may be relevant in other settings using recyclable and non-recyclables, and particularly problematic in a food storage setting. Further, during the processing of recyclable materials such as cardboard and some plastics the recycling process will substantially reduce the problem of pathogens, however, for other recyclable products such as glass it is important to reduce scenarios where persons that are involved in the recycling process including end users are deterred due to bad smells or to other factors due to a lack of sanitation (e.g. where have bacterial leaching in recycled material used in roads, or other building or construction projects).

Furthermore, one or more of the defects or failings discussed above are also readily apparent in the food processing or packaging industry, where the cleanliness or sanitation of equipment used in the conveying and/or in the storage of food materials is an increasing concern among food manufacturers, food packagers and the like (e.g. managing the presence of bacteria such as listeria, or other pathogens commonly found on food). In particular, in the past, food processing or packaging areas have used toxic, harmful or irritating cleaning or sanitising products in the cleaning and sanitising of machinery or the working environment, requiring rinsing after application, personnel applying the products to be dressed in high level PPE, and/or requiring that workers leave a work area during cleaning or sanitising.

The need for effective sanitising, and related or associated benefits associated with control of dust and odour, can be understood as relevant in a variety of settings in industrial, commercial, institutional and other facilities, and particularly where the cleaning or sanitising of machinery, equipment, apparatus or working environment more generally (e.g. floors, railings, doors, etc.) is important.

Therefore, there is a need for improved systems of cleaning or sanitising material that obtain one or more of the following:
cleaning or sanitising of material on an industrial scale, while conserving the use of cleaning or sanitising agents; and/or
cleaning or sanitising of equipment used to transport or of equipment used in manufacturing or processing of material where sanitisation is important, while conserving the use of cleaning or sanitising agents; and/or
application of the cleaning or sanitising agent for effective cleaning of the material without the need for high temperature application; and/or
application of the cleaning or sanitising agent for complete or substantially complete removal of pathogens over long periods of time; and/or
providing a non-hazardous or nontoxic sanitiser; and/or
providing a food-safe sanitiser effective in the context of food packaging or food processing; and/or
providing more cost-efficient methods of applying sanitiser on a large scale; and/or
providing systems and methods of sanitising that reduce disruption to work areas for cleaning purposes; and/or
providing a means to control and remove listeria and other foodborne pathogens in food packaging or food processing environments; and/or
control of dust, including by particle suppression; and/or
reduction or management of material odour; and/or
reduction or management of pests that are attracted to the material; and/or
providing for improved recycling of more difficult to sort or categorize recyclables.

The present invention seeks to systems and methods of sanitising, which will overcome or substantially ameliorate at least some of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY

According to a first aspect of the invention there is provided a method of cleaning or sanitising a material, the method including the step(s) of spraying the material with a sanitising liquid.

Preferably the spraying is provided as the material travels along a conveyor path and/or falls therefrom using one or more liquid spraying devices.

Preferably, spraying the material comprises pressure spraying, misting, foaming and/or fogging.

Preferably, the material comprises one or more of the following:
Recyclable material such as crushed glass or plastics;
Aggregate material;
Organic or partly organic material;
Processed food;
Food producing trees; and/or
Agricultural produce.

According to a second aspect of the invention there is provided a method of cleaning or sanitising a material, the method including the following steps:
Spraying the material with a sanitising liquid as it travels along a conveyor path using one or more liquid spraying devices;
Spraying the material with the sanitising liquid as it exits or falls from the conveyor path one or more liquid spraying devices;
Spraying the material with the sanitising liquid as it forms a pile below the conveyor path.

According to a third aspect of the invention there is provided a method of cleaning or sanitising a material, the method including the steps of:
Providing one or more liquid spraying devices adapted to spray a sanitising liquid;
Connecting the one or more liquid spraying devices to a sanitising liquid supply;
Providing a conveyor path for conveying the material;
Delivering the sanitising liquid to the one or more liquid spraying devices to spray the material with the sanitising liquid as it travels along the conveyor path and/or it exits or falls from the conveyor path.

According to a fourth aspect of the invention there is provided a system for cleaning or sanitising a material, the system including:

one or more liquid spraying devices for spraying the material with a sanitising liquid; and a conveyor path;
wherein the one or more liquid spraying devices spray the material as it travels along the conveyor path and/or falls therefrom.

According to a fifth aspect of the invention there is provided a system for cleaning or sanitising a material, the system including one or more liquid spraying devices adapted to:
spray the material with a sanitising liquid as it travels along the conveyor path;
spray the material with the sanitising liquid as it exits or falls from the conveyor path; and/or
spray the material with the sanitising liquid as it forms a pile below the conveyor path.

Preferably, the system includes a sanitising liquid delivery line to deliver the sanitising liquid to the one or more liquid spraying devices.

According to a sixth aspect of the invention there is provided a system for cleaning or sanitising a material, the system including:
one or more liquid spraying devices adapted to spray a sanitising liquid, the one or more liquid spraying devices being connectable to a sanitising liquid supply;
a conveyor path for conveying the material;
a sanitising liquid delivery line to deliver the sanitising liquid to the one or more liquid spraying devices to spray the material with the sanitising liquid as it travels along the conveyor path and/or it exits or falls from the conveyor path.

According to a seventh aspect of the invention there is provided a method of cleaning or sanitising conveying equipment and/or storage areas or surfaces the method including the steps of spraying the equipment and/or storage areas or surface using one or more liquid spraying devices for spraying a sanitising liquid.

Preferably, the one or more spraying devices comprise misting or fogging devices located in an upper region of a storage area. Preferably, the misting or fogging devices are located along a misting or line located in the upper region of the storage area.

Preferably, the conveying equipment comprises material crushers and the means of conveying the material to the crushers.

According to an eighth aspect of the invention, there is provided a system for cleaning or sanitising aggregate material (for example, contaminated soils or rocks, but may also be glass), the system including:
a. a large volume tank including a diluting liquid, to which sanitising liquid is added;
b. spraying the aggregate material with one or more liquid spraying devices;
c. periodically moving the sprayed aggregate between a plurality of sieves, screens, sink float tanks, and/or shakers to separate and permit further spraying of the aggregate material.

Preferably, the aggregate materials are separated by running through a rotating barrel.

Preferably, separated product is sprayed with sanitising liquid at high pressure.

Preferably, the large volume tanks are up to or around 800,000 Litres in volume, or more. Preferably, an initial dosing of the tank is to provide a dilution ratio of 1:200 (i.e. 4,000 L of sanitising liquid for a tank of 800,000 L). Preferably, the tank is topped up with a further amount of diluted sanitising liquid periodically (e.g. every week, for 4-6 weeks) in accordance with the amount of liquid that is being sprayed (e.g. the capacity of one large volume tank being sprayed per period of time, or one week) to maintain adequate levels of diluted sanitising liquid in the tank.

According to a ninth aspect of the invention, there is provided methods and systems of cleaning or sanitising surfaces. Preferably, the methods and systems are for cleaning hard surfaces.

Preferably, the surfaces comprise parts of machinery or equipment, or other surfaces in a manufacturing, processing or other industrial facility.

According to further aspects of the invention, there are provided systems for cleaning or sanitising material; including any one or more of the following:
Maintaining sanitisation of drainage areas, to reduce the likelihood of pathogens being blown out of drains onto surfaces (e.g. where a pathogen survives and grows on organic matter trapped in a drain, water is flushed through the drain, and the pathogen aerosolises and travels away from the drain area);
In the sanitising of filters of air blowers that are used to push material along conveyors, wherein a filter pad soaked in sanitising liquid is placed at or near the air inlet of the blower;
Application of sanitising liquid to conveyers, hoppers and bins that distribute and hold food products (for example, processed meats, vegetables, seafood, or dairy), wherein application is by a spray bar positioned near the conveyor, hopper or bin;
Clean-in-Place sanitation for dairies, where sanitising liquid is recirculated around piping, vessels, filters, tanks and fillers and other areas and equipment to be cleaned. Preferably, spray and/or fog nozzles are used in the application of the sanitising liquid;
Foaming cleaning of floors in food processing plants, food outlets including retail or other areas where food is processed, bought, sold, stored or transported;
Sanitising of hard surfaces in industrial, commercial and other facilities, and machinery, equipment or apparatus used therein;
Sanitising of other hard surfaces including desks and countertops.

Preferably, the systems and methods of the invention are adapted to be applied to large scale sites or working areas.

Preferably, spraying the material with sanitising liquid comprises spraying with a foamed sanitising liquid and/or a misted liquid and/or a fogged liquid. Preferably, providing one or more liquid spraying devices adapted to spray a sanitising liquid comprises providing one or more liquid foaming devices adapted to spray a foamed sanitising liquid and/or one or more liquid misting devices adapted to spray a sanitising liquid mist and/or one or more liquid fogging devices adapted to spray a fogged sanitising liquid.

Preferably, the sanitising liquid comprises wetting agent properties or surfactant properties to enable the sanitising liquid, particularly when fogged, to operate as a dust suppressant (e.g. for fugitive dust) or as a suppressant of suspended particulate matter (e.g. aerosolised pulverised glass).

Preferably, the wetting agent or surfactant properties provide improved coverage of the sanitising liquid on the material or surface to be sanitised. Preferably, the wetting agent or surfactant properties enable the sanitising liquid to attach to and spread around the material or surface.

Preferably, the sanitising liquid can be diluted to a low concentration (e.g. 1:100, 1:200, 1:400 or lower sanitising liquid: diluent concentrations) while remaining effective in some settings.

Preferably, the sanitising liquid enables bonding of the sanitising liquid (e.g. the fatty acid:amino acid mixture) to the pathogen to enable it to adhere to the pathogen and provide long lasting sanitisation.

Preferably, the spraying with sanitising liquid provides a means for settling the material when the material is in a dust particle form. Preferably, the dust particle material is settled by overhead sprayers (e.g. foggers or misters) that operate on a timer. Preferably, the timer sprays the sanitising liquid once every predetermined period of time (e.g. every hour, or every multiple hour block) for a predetermined period of time (e.g. between about 1 minute and 5 minutes, and more preferably 2 minutes) over a predetermined area (e.g. an entire internal production/storage area) to settle dust particle material and inhibit and/or reduce pathogens. Preferably, such operation therefore also provides a means to consequently manage malodours.

Preferably, fogging the sanitising liquid includes pressurising the liquid through nozzles designed to produce small dimension droplet particles, also known as ‘fog’ droplets (e.g. approx. 1-10 microns, often classified as Extremely Fine; this can be compared to ‘mist’ droplets which may be created by using misting nozzles to produce droplets having a diameter of approx. 10-100 microns, often classified as Fine or Very Fine). The fog may evaporate into the atmosphere within a short period of time (typically, seconds). The fog provides benefits relevant to the invention, including:
Means of delivering the sanitising liquid to the material which may include surfaces of equipment and facilities, floors, drains etc. and controlling any airborne microorganisms;
Encapsulating and/or suppressing dust particles in the air as they fall to the ground;
Permitting a lower total amount of sanitising liquid to be used, per unit of time. For example, a much lower flow rate may be utilised compared to misting or spraying, which means that higher concentration rates may still be used during fogging (and the benefits of higher concentration rates achieved, such as a faster reduction of pathogen growth or pathogen elimination) without a substantial cost burden associated with high sanitising liquid usage;
Increasing the ability for the sanitising liquid to attach to the material. Fogging atomises the sanitising liquid to a very fine droplet which, together with the bonding capacity of the specified sanitising liquid (e.g. fatty acid:amino acid mixture), provides an effective coating of the material, product or surface to be sanitised and thereby improved inhibition of pathogen growth (or complete removal of the pathogen).
Due to the very small droplet size, fogged sanitising liquid is able to penetrate more deeply within material, products or surfaces to be sanitised (e.g. by penetrating small dimension crevices, cracks or gaps in the material, or an upper or outer layer of the material). Pathogens are prone to survive and grow in such areas, and they may be more difficult to reach with sprayed or misted sanitising liquid, as opposed to when they are fogged.

Preferably, the sanitising liquid is a composition adapted to interact with the pathogen on a nanoscale.

Preferably the sanitising liquid is adapted to inhibit growth of and/or eradicate one or more of the following pathogens: gram-negative bacteria, gram-positive bacteria, listeria monocytogenes, foodborne pathogens, Methicillin-resistant Staphylococcus aureus (MRSA), E.coli, fungus, Mycobacteria, pneumoniae bacteria, and/or virus.

The sanitising liquid may be a composition comprising a mixture of a fatty acid:amino acid. Preferably, the mixture is in a molar ratio between about 1:0.6 to about 1:1.6.

Preferably, the fatty acid of the sanitising liquid is any one or more of a C4 to C40 fatty acid.

Preferably, the fatty acid is one or more is one or more of: Octanoic acid, Nonanoic acid, Decanoic acid (capric acid), Undecylenic acid, Laurie acid, Tridecylic acid, Myristic acid, Linoleic acid, Arachadonic acid and Arachidic acid.

Preferably, the amino acid of the sanitising liquid is an amino acid having an electrically charged basic side chain.

Preferably, the amino acid is one or more of L-Arginine, Histidine and Lysine.

Preferably, the sanitising liquid is a composition comprising an aqueous liquid.

Preferably, the sanitising liquid composition comprises a mixture of decanoic acid:L-Arginine.

Preferably, the sanitising liquid composition forms a complex of decanoic acid and Arginine having a molar ratio of between 1:0.6 to 1:1.6. Preferably, the composition comprises at least 0.001% w/w of the complex of decanoic acid:Arginine. Preferably, the molar ratio of decanoic acid:Arginine is in approximately 1:1 molar ratio. Preferably, the composition is substantially free of cetyl alcohol and/or Rhein.

Preferably, the sanitising liquid composition comprises an aqueous solution of a complex of decanoic acid:L-Arginine having a molar ratio of decanoic acid:L-Arginine of between 1:0.6 to 1:1.6 at a total concentration of at least about 0.001% w/w, wherein the composition is substantially free of cetyl alcohol.

Preferably, the sanitising liquid composition includes active ingredients in a range between about 0.1% and about 10% w/w. It is preferred that the composition includes active ingredients in a range between about 0.5% and about 8% w/w. It is particularly preferred that the composition includes active ingredients in a range between about 1% and about 5% w/w.

Preferably, the decanoic acid is between 0.1% and 10% w/w (e.g., between about 0.5% and about 5%, between about 1.0% and 5%, about 1.5%, etc.).

Preferably, the Arginine (e.g., L-Arg) is between about 0.1% and 10% w/w (e.g., between about 0.5% and about 5%, between about 1.0% and 5%, about 1.52%, etc).

Preferably, the sanitising liquid composition includes: water, L-Arginine, decanoic acid and an emulsifier/solubiliser such as Polysorbate 20, Polysorbate 80, Polysorbate 60, Poloxamer, Emulsifying wax, Sorbitan monostearate, Sorbitan monooleate, Sodium lauryl sulfate, Propylene glycol monostearate, Diethylene glycol monoethyl ether, Docusate sodium, etc. The composition may include a fragrance (e.g. eucalyptus, lemon, etc.).

Preferably, the composition comprises benzalkonium chloride. Preferably, the composition includes benzalkonium chloride at a final concentration of between about 0.05% and about 5% w/w (e.g., between about 0.05% and about 0.5%, between about 0.1% and 0.5%, about 0.13%, about 0.4%, etc.).

Alternatively, the sanitising liquid may be a composition comprising a mixture of L-Arginine, undecylenic acid and cassic acid.

The sanitising liquid may be a composition comprising a mixture of L-Arginine, undecylenic acid and cassic acid, wherein the percentage of cassic acid in the mixture is greater than 0.03% w/w of the mixture. Preferably, the percentage of cassic acid in the mixture is between 0.03% and 2.3% w/w of the mixture, the percentage of L-Arginine is between 47% and 73% w/w of the mixture, and the percentage of undecylenic acid in the mixture is between 26% and 53% w/w of the mixture. Preferably, the cassic acid is encapsulated in the undecylenic acid.

Preferably, the sanitising systems and methods inhibit pathogen (e.g. bacteria) growth in or on the material, surface, etc. It is particularly preferred that the sanitising systems and methods enable elimination of pathogens in or on sanitised material, surface, etc.

The composition is preferably able to operate to inhibit pathogen growth over long periods of time, such as weeks or months.

Since the proposed sanitising liquid is food-safe and non-toxic, it may be used continuously even when the area to be sanitised is occupied by people (e.g. workers in food and dairy processing or packaging areas).

The composition is preferably non-corrosive and non-caustic. Preferably, the composition does not in operation degrade the materials in the dispensing system or cause blockages in the liquid dispensing apparatus such as the nozzles and the jets.

Preferably, the material comprises recyclable components. Preferably, the material comprises recyclable components in aggregate, concrete (e.g. crushed concrete), cement, silt, grit, rail ballast, rocks (e.g. screening rocks or landscape rocks) or material used in building and construction. In a particularly preferred form, the material comprises blended products such as road base. Preferably, the material comprises fill materials such as ripped blue shale or soil. Preferably, the material comprises recycled brick and tile, such as mixed masonry fines, crushed brick and tile fines or aggregate, or brick rubble. Preferably, the material comprises recycled asphalt.

Preferably, in some contexts such as recyclable glass or some plastics, the material comprises small segments. The small segments may have a maximum thickness of approximately 40 mm or less. The small segments may have a maximum thickness of approximately 20 mm or less. The small segments may have a maximum thickness of 10 mm or less. It is particularly preferred that the small segments have a maximum thickness of 5 mm or less. It will be understood that in other context, such as recyclable asphalt, brick rubble or rocks, the size of segments aggregate material may be greater than 40 mm.

The material may comprise glass material. Preferably, the material comprises crushed glass. Alternatively, the material may comprise one or more, or a combination of non-fragmented (i.e. intact) or fragmented (i.e. compressed, crushed, pulverised, or otherwise broken down) materials such as plastic, steel, aluminum, glass, or other hard recyclable materials.

Alternatively, the material comprises hard surfaces or other surfaces such as metals, tiles, rubber, or surfaces of machinery or equipment.

In some applications, the material may comprise agricultural produce such as fruit and vegetables, or processed or packaged food of any kind.

In some applications, the material may comprise fruit or other food producing trees. Preferably, the sanitising liquid is adapted to treat fungal infections on trees, and in particular citrus trees. In a preferred form, the sanitising liquid is adapted to treat Greasy Spot Fungus, Citrus Melanose, Scab Disease and/or Foot Rot.

In some applications, the material may comprise surfaces of vehicles used for transportation of food.

Preferably, the one or more liquid spraying devices are adapted to coat the entire surface area, or substantially the entire surface area, of the material with the sanitising liquid.

Preferably, there may be used a variety of nozzle heads to control the volume of sanitising liquid that is used during spraying. Preferably, the nozzle heads are colour coded for ease of use.

Preferably, the nozzle heads provide a flow rate in a range between about 0.15 gallons per minute (or about 55 mL per minute) to 1.0 gallons per minute (3.85 L per minute).

Preferably, the one or liquid spraying devices are adapted to provide a foamed spray of the sanitising liquid.

Foaming may be achieved by foaming means. The foaming means may include running the sanitising liquid at pressure through a foaming device, including a foaming nozzle including a sieve or other means conducive to producing foaming, to produce the foamed sanitising liquid. For foaming, the pressure is preferably mains pressure. Foaming nozzles or systems incorporating them may also include air injection.

Preferably, the foaming nozzles include orifices having diameters between approximately 0.5 and 3 mm. In a preferred form the foaming nozzle orifice has a diameter in a range between about 1 mm and 1.5 mm.

Preferably, the foaming devices comprise foaming nozzles that include an air intake, so that sanitising liquid is foamed as it passes through the nozzle.

Foaming devices may comprise manually operated foaming wands or foaming guns.

Fogging may be achieved by fogging means. The fogging means preferably includes running the sanitising liquid at high pressure though a fogging nozzle is preferably in a range between 800 and 1200 psi. It is preferred that the pressure is in a range around 1000 psi.

Preferably, the fogging nozzles have orifices having diameters between approximately 0.1 mm and 2 mm.

Preferably, the fogging nozzles have a flow rate in a range between 0.05 L/min and 6 L/min. It is particularly preferred that the fogging nozzles have a flow rate in a range between 0.1 L/min and 0.5 L/min.

The foamed and/or fogged sanitising liquid may provide for increased contact time on the material as the foamed and/or fogged sanitising fluid is able to remain on the material longer than would be the case if the sanitising liquid was applied in liquid form.

The foamed sanitising liquid may provide means for visual indication of where it has been applied and where it has not, and thereby improved sanitising due to better coverage.

Preferably, foaming application substitutes liquid application by conventional spraying due to suitability to certain applications as described herein, and thereby uses between about 1% to 5% of a comparable amount of sanitising liquid to perform substantially same sanitising as conventional spraying (e.g. where droplet diameter is greater than 100 microns).

Preferably, fogging application uses between about 10% to 20% of a comparable amount of sanitising liquid to perform substantially the same sanitising as foaming (i.e. only about 0.1% to 1% of the amount of sanitising liquid to perform substantially the same sanitising as conventionally sprayed sanitising liquid).

In this manner, it will be understood that foaming application, and more particularly fogging application, reduces costs of the sanitising liquid composition required to perform a given application. This also provides scope for increasing the concentration of the sanitising liquid when diluting, and thereby obtaining improved cleaning and sanitisation, while remaining within a comparable budget for liquid sanitising liquid application.

Preferably, for systems that are located partly or entirely outdoors or not in an enclosed environment, misting or conventional spraying application, or other drift reduction spraying methods, are preferred. Preferably, the droplet size of the sprayed liquid is larger for outdoor settings to give the sanitising liquid greater weight, and thereby reduce impact by wind or other environmental conditions. Fogging may be applied in outdoor settings where the sprayer is located proximate to the surface to be sanitised.

Where the material is loose material that is piled, heaped or otherwise amassed, whether on a ground or conveying surface or similar, the spraying devices preferably comprise spray jets, conventional sprayers or similar spraying devices that enable the sprayed sanitising liquid to filter into lower portions of the piled, heaped or massed material.

Preferably, where just a surface layer of the material, surface or produce is required to be sanitised, fogging and/or foaming is applied.

Combinations of fogging, and spraying with spray jets, conventional spraying, misting and/or foaming, may be provided in environments where the complementary benefits of different spraying devices is able to be realised.

Preferably the spraying devices are positionable to spray receptacles, bins vessels, material on or falling from conveyors, hoppers, transfer points, filters, tanks and fillers and other equipment or machinery.

Preferably, there are a plurality of spraying devices along one or more sanitising liquid delivery line(s). Preferably, the spraying devices (e.g. misting, foaming and/or fogging nozzles) are spaced apart in regular intervals along the delivery line(s). Preferably, the spacing is in a range between about 0.5 meters and 2 meters. In a particularly preferred form, the spacing is approximately every 1 meter. For spraying larger areas, a plurality of liquid delivery lines may be utilised.

The system is preferably adapted to be retrofit to an existing liquid delivery system, including to one or more, or a part of one or more, liquid delivery lines and/or liquid spraying devices of the existing system.

Preferably, there is provided a controller for controlling the delivery of sanitising liquid. Preferably, the controller is adapted to control one or more diluting units, control valves, high pressure pumps, isolation valves, or other components used for the delivery of sanitising liquid in accordance with the systems and methods of the invention.

Preferably the controller is adapted to control one or more of the following parameters:
Dosing or concentration (e.g. where sanitising liquid is mixed with a diluent such as water). Preferably, the system incorporates a controllable diluting device;
Timing of delivery of sanitising liquid to the spraying devices;
The duration of delivery of sanitising liquid to the spraying devices;
Which spraying devices on which delivery lines are delivered sanitising liquid.

Preferably, the timing of delivery and duration of delivery is controllable by controllable valves. Preferably, the controllable valves are solenoid valves.

Preferably, the controller is adapted to independently control a plurality of controllable diluting devices and delivery of the sanitising liquid to the spraying device and/or along the delivery lines, so that spraying devices on a plurality of lines can be delivered a plurality of (different) concentrations of sanitising liquid, at a plurality of different times and for a plurality of different durations of time. Preferably, the plurality of delivery lines are connected to a plurality of spraying devices in a plurality of different locations (e.g. different zones in a work area or worksite), where delivery of sanitising liquid to the plurality of spraying devices is independently controllable by the controller. Preferably, for example, delivery of sanitising liquid to a first delivery line supplying a first zone foaming spray bar is independently controllable to a second delivery line supplying 100 fogging nozzles for fogging a second zone, and delivery to the first and second zones is independently controllable to delivery to a third delivery line that includes 10 misting nozzles for delivery of misted sanitising liquid to a third zone, etc. Alternatively, the different zones may all be supplied sanitising liquid having the same concentration, which may be supplied from a single diluter. It is also contemplated that all spraying devices may be of the same type (e.g. all fogging, or all conventional spraying devices)

Preferably the controller is programmable to enable adjustable control of the parameters over a set period of time (e.g. so that dosing rates and/or the zones that are sanitised and/or the length of sanitising time during a cycle, for example, can be programmed to vary over the course of one day). Preferably, the controller is a programmable logic controller. Alternatively, the controller may be a personal computing device.

Preferably, the sanitising system includes one or more of, or a combination of, each of the following: filtration devices, high pressure pumps (e.g. for fogging), automatically controllable valves (e.g. high-pressure solenoid valves for zoning, timing and duration of application, controllable by the controller), isolation valves (for isolating parts of the system for maintenance, updating or cleaning), a diluter source (e.g. mains water or water tank), piping or other distribution means such as tubing for delivery of sanitising liquid to the spraying devices, connecting parts such as Tees, elbows and other connectors, hanging or suspension components to hold delivery lines in an elevated location, diluting units (which may be controllable by the controller), and mixing chambers (e.g. to provide turbulent mixing of sanitising liquid and diluent), and other components used in systems for delivering sprayed liquid as known to the skilled addressee.

Preferably, the sanitising liquid delivery line(s) are connected to the diluter(s) and/or other components.

Preferably, the controller is connected to the controllable devices/components. Connection to the devices/components may be via a wired or wireless connection.

Where the system includes, or is adapted for spraying material on or from a conveyor, the conveyor may be a belt. Alternatively, the conveyor may be any other means of transporting the material, such as a slide, chute, hopper, or rocking container. Preferably, the conveyor is a cleated conveyor.

In an illustrative embodiment, the conveyor path may carry the material along the path at a rate between about 0.1 meters per second and 0.5 meters per second. Depending on the dimensions of the facility and conveying equipment, etc, the conveyor rates may vary.

In an illustrative embodiment, the collective flow rate of the one or more liquid spraying devices is between about 0.1 liters to 10 liters per second. Depending on the scale of the system, the collective flow rates may vary.

Preferably, the material on the conveyor path is between about 1 cm to about 5 cm deep. It is preferred that the material on the conveyor path is between about 3 cm to about 5 cm deep.

Preferably, the conveyor path width is between about 40 cm and 1 meter. It is preferred that the conveyor path width is between about 50 cm and 70 cm.

The conveyor path may be defined by a surface of a conveyor belt. The conveyor belt may be wider than the material conveyor path.

In an illustrative embodiment, the conveyor path length is between about 10 and 30 meters. It is preferred that the conveyor path length is between about 15 and 25 meters.

In an illustrative embodiment, the conveyor path is between about 7 to 8 meters above a floor surface where the material exits the conveyor path. Preferably, once exiting the conveyor path the material forms in a pile, mound or large localised mass on the floor surface, below the conveyor path.

Preferably, the pile accretes to a height between about 5 to about 10 (or more preferably, between about 6 to about 7) meters during material sanitization.

Preferably, the pile expands to a base having a diameter of between about 5 to about 10 (or more preferably between about 6 to about 7) meters during the material sanitization.

Alternatively, where the material is sanitised out of doors or in very large indoor or otherwise protected areas, the material may be conveyed such that to falls to a ground surface or on top of existing material. The conveyor path may fall upwards of 20 meters to the ground surface or to the top of existing material, and it may form a pile with a base having corresponding larger diameter.

Preferably, the one or more spraying devices are adapted to spray the pile as it accretes below the conveyor path.

In an illustrative embodiment, during 8 hours of material sanitization, the pile accretes to a height between about between about 5 to about 10 (or more preferably between about 6 to about 7) meters during the material sanitization, the pile having a base diameter between about 5 to about 10 (or more preferably between about 6 to about 7) meters. Preferably, the pile accretes to a pile having the aforesaid dimensions two or three times per 8 hours.

In an illustrative embodiment, the conveyor path is adapted to carry between about 1500 tonne and 2000 tonne of material per 8 hours. It is preferred that the conveyor path is adapted to carry between about 1600 tonne and 1800 tonne of material per 8 hours.

Preferably, the one or more liquid spraying devices are adapted to spray in a spray zone along the conveyor path.

Preferably, the one or more liquid spraying devices comprise two or more liquid spraying devices having overlapping spray zones.

Preferably, the one or more liquid spraying devices comprise two or more liquid spraying devices having overlapping spray zones on the conveyor path.

Preferably, the one or more liquid spraying devices comprise one or more fan jets (i.e. flat fan or wide flat fan nozzles) positioned to spray the material with the sanitising liquid as it travels along the conveyor path. Alternatively, or in combination, the one or more liquid spraying devices may comprise deflector nozzles and/or elliptical orifice nozzles, or other jet nozzles known to the skilled addressee. Preferably, the one or more jets may comprise two or more jets having one or more overlapping spray zones along the conveyor path. The one or more jets may comprise 3 or 4 jets providing 2 or 3 overlapping spray zones.

The overlapping spray zones are preferably partly, but not entirely overlapping.

One or more of the one or more spraying devices may (alternatively, or in addition to the one or more spraying devices adapted to spray along the conveyor path) be arranged to surround, or substantially surround, the material as it exits the conveyor path.

One of more of the one or more spraying devices be adapted to spray along the conveyor path and to spray the material as it exits the conveyor path.

Preferably, the one or more liquid spraying devices comprise three or more liquid spraying devices having overlapping spray zones adjacent to and below the conveyor path.

Preferably, the one or more spraying devices adapted to spray material as it exits and/or falls from the conveyor path, are arranged such that the material passes through a region defined by the one or more spraying devices. Preferably, the region is defined by a ring or partial ring including the one or more spraying devices. Preferably, the region is substantially circular or oval shaped. Alternatively the ring may include a plurality of straight line segments.

The diameter of the substantially circular region, or major axis of the oval shaped region, may be between about 0.5 meters and about 1.2 meters. Preferably, the diameter of the substantially circular region, or major axis of the oval shaped region, is between about 0.7 meters and about 1 meter.

It is preferred that the ring or partial ring includes three or more spraying devices. Preferably, the ring or partial ring is connected to part of a conveyor providing the conveyor path.

Preferably, the one or more spraying devices are arranged to produce a substantially cylindrical (elliptic or circular cylindrical) or substantially conical spray zone through which the material passes as it exits the conveyor path. Preferably, the diameter of the cylindrical or conical spray zone is between 5 to 10 meters. It is particularly preferred that the diameter of the zone is between 6 to 7 meters. Preferably, the diameter of the cylindrical or conical spray zone extends to a floor surface. Preferably, the diameter of the cylindrical or conical spray zone extends to cover the pile, mound or mass of material forming below the conveyor path.

Preferably, there are a plurality of rings or partial rings, whereby the inner ring(s) deliver a spray zone that has a smaller diameter than the outer rings. Preferably, the inner ring(s) are controllable such that the inner rings are activated before the outer ring(s). Preferably, activation of the plurality of rings is controlled by a controller to coincide with the increasing diameter of the pile of material forming below the conveyor path.

Preferably, the one or more spraying devices are arranged to spray the material as it falls downwardly from the conveyor path. Preferably, the one or more spraying devices are arranged to spray the material as it rotates while falling downwardly from the conveyor path. Preferably, the one or more spraying devices are arranged to spray the upper layer of material as it forms on the pile below the conveyor path.

Preferably, the one or more spraying devices are arranged to spray the material for all, or substantially all, of the time that it is falling downwardly from the conveyor path and/or the upper surface of the material when it is in a pile formed below the conveyor path.

Preferably, the one or more spraying devices are arranged to spray the material as it falls downwardly from an end of the conveyor path.

Preferably, the one or more liquid spraying devices comprise one or more spray jets adapted to spray the material as it exits or falls from the conveyor path. Preferably, the one or more spray jets comprise one or more cone (or hollow cone) spray jets. Alternatively, or in combination, the one or more spray jets may comprise spiral nozzle jets, misting nozzle jets, or other nozzle jet types known to the skilled addressee.

Preferably, the one or more liquid spraying devices comprise one or more spray rings and/or one or more spray bars. Preferably, the spraying devices are adapted to spray the material as it exits and/or falls from the conveyor path.

Preferably the one or more liquid spraying devices comprise one or more spraying devices adapted to spray the material as it travels along the conveyor path and one or more spray spaying devices adapted to spray the material as it exits and/or falls from the conveyor path and/or as it forms a pile below the conveyor path.

In some applications, it is preferred that spraying with fogged sanitising liquid causes a layer of fogged sanitising liquid to form on the material as it travels on a conveyor.

Preferably, the collective flow rate of the one or more spraying devices adapted to spray the material as it exits and/or falls from the conveyor path is greater than the collective flow rate of the one or more spraying devices adapted to spray the material as it travels along the conveyor path.

Preferably, the conveyor path is arranged such that the material rotates within the spray zone as it exits and/or falls from the conveyor path.

Preferably, the method of sanitising or cleaning further includes the step of providing the sanitising liquid supply. Likewise, the sanitising or cleaning system may include the sanitising liquid supply. The sanitising liquid supply may comprise a tote or other large container adapted to hold the sanitising liquid. Preferably, the sanitising liquid supply comprises a 1000 liter tote.

Preferably, the method includes the step of dispensing a controlled amount of the sanitising liquid using a measuring unit.

Preferably, the method includes providing the measuring unit. Likewise, the sanitising or cleaning system may include a measuring unit for dispensing a controlled amount of the sanitising liquid using a measuring unit.

Preferably the measuring unit includes communication circuitry to wirelessly communicate flow related data to the controller and/or a personal electronic computer.

The measuring unit is preferably a Bintech® unit.

The method preferably includes the step of diluting the sanitising liquid with a diluting liquid to form a diluted sanitising liquid to be delivered to the one or more spraying devices. Preferably, the diluting step is prior to delivering the sanitising diluted liquid to the one or more spraying devices.

Preferably, the diluting step provides a diluted sanitising liquid comprising a mixture of sanitising liquid: diluting liquid in a ratio of between about 1:10 to 1:500.

In some settings, for example, food processing or food packaging areas, where fast acting and/or continuous and robust pathogen reduction or elimination is desired, it is preferred that the diluting step provides a diluted sanitising liquid comprising a mixture of sanitising liquid: diluting liquid in a ratio of between about 1:10 to 1:50. In such settings, use of spraying devices comprising foggers may be preferred to reduce the total amount of sanitising liquid used (compared to conventional sprayers, misters or even foaming devices) so that higher concentrations of sanitising liquid can be delivered without raising the cost burden associated with the sanitising liquid. Alternatively, where fogging is not practical, such as where only localised application is required (e.g. for the drains of an indoor food processing area), or in outdoor environments, spraying of diluted sanitising liquid diluted within the abovementioned range may be preferred. Alternatively, lower concentration dilution may also be applied in the context of food processing or food packaging areas, as a further measure to manage the cost of sanitising liquid.

In some settings (for example, large scale facilities such as transfer stations for recyclable materials), it is preferred that the diluting step provides a diluted sanitising liquid comprising a mixture of sanitising liquid: diluting liquid in a ratio of between about 1:100 to 1:300. Is it particularly preferred that a dilution ratio between about 1:150 and 1:250, and more particularly still, around 1:200, is applied for sanitising aggregate recyclable material with fogged sanitising liquid (for example, crushed glass, at an indoor recycling station).

In some settings (for example, sanitising of outdoor piles of material) is preferred that the diluting step provides a diluted sanitising liquid comprising a mixture of sanitising liquid: diluting liquid in a ratio of between about 1:200 to about 1:300 (and even more particularly around 1:250); alternatively dilution may be in a higher concentration range between 1:50 and 1:200 where the cost of the sanitising liquid composition does not prohibit using a higher concentration of active ingredients in the diluted sanitising liquid. In outdoor settings, it is preferred to jet or conventional spray the sanitising liquid to minimise spray drift that results from winds or other environmental acts.

For sanitising organic or partly organic material such as aggregate rocks or soil, or crushed glass, the preferred dilution range is 1:200, or more broadly in a range between 1:150 and 1:250.

For sanitising agricultural produce, the preferred dilution range is 1;300, or more broadly in a range between 1:200 and 1:400. Systems for sanitising produce may apply conventional spraying or misting on conveyors, or other equipment for transporting or processing produce, in a manner similar to the sanitising of aggregate recyclables (e.g. crushed glass).

Preferably, the method includes providing a diluter for diluting the sanitising liquid with the diluting liquid. Likewise, the sanitising or cleaning system may include a diluter for diluting the sanitising liquid with the diluting liquid.

Preferably, the diluting liquid is water.

Preferably, the diluter is water driven such as a venturi diluter. Alternatively, it may be an electronic or mechanically actuated diaphragm metering pump, or a peristatic metering pump. It is preferred that the diluter is fluidly connected to a diluting liquid supply and the sanitising liquid supply.

Preferably, the diluter is able to dilute the sanitising liquid to a concentration in a range between 1:10 and 1:500, or any suitable sub-range or particular concentration within those ranges.

Preferably, the diluter is automatically controllable to provide an adjustable dilution rate.

Preferably, the diluter is fluidly connected to the measuring unit. Alternatively, the diluter may incorporate the measuring unit.

Preferably, in an outdoor application, the method provides spraying between about 20,000 and about 40,000 liters of the diluted sanitising liquid per 8 hours. It is preferred that the method provides spraying between about 28,000 and about 36,000 liters of diluted sanitising liquid per 8 hours. Likewise, it is preferred that the system is configured to spray the aforesaid of diluted sanitising liquid amounts within a period 8 hours.

Preferably, the diluting liquid supply is a tank.

It is preferred that the diluting liquid supply is a water tank with a capacity of approximately 30,000 to 50,000 liters. Preferably, the diluting liquid supply is a water tank with a capacity of approximately 40,000 liters.

Preferably, the tank includes a lid.

Preferably, the tank is continuously replenished with the diluting liquid.

Preferably, the diluting liquid supply is connected to a diluting liquid supply source.

Preferably, the diluting liquid supply source is mains water, dam water, or run off water from the roofs, etc. of buildings.

Preferably, the diluting liquid is filtered.

The method may include filtering the diluting liquid prior to it entering and/or after it exits the diluting liquid supply.

Preferably, the method includes providing a filter to filter the diluting liquid prior to it entering and/or after it exits the diluting liquid supply.

Preferably, the method includes the step of filtering the diluting liquid using a 20 micron filter, and/or a 10 micron filter, and/or a 5 micron filter to remove solids (and in particular, to inhibit blockages forming in the spraying devices). The filter may be replaced weekly, or at another regular interval. Preferably, the filters are not so fine that they require daily or more regular replacement.

Likewise, a preferred system of the invention may include the one or more of the aforesaid filters to filter the diluting liquid prior to it entering and/or after it exits the diluting liquid supply.

Preferably, the method includes pumping the diluting fluid from the diluting fluid supply to the diluting unit and/or from the diluting unit to the conveyor path.

The method of the invention preferably includes providing a pump adapted pump liquid at the desired flow rate at a pressure between about 60 psi or 500 Newton per square meter and about 120 psi or 1000 Newton per square meter. Alternatively, in high-pressure applications there is provided a high pressure pump adapted to pump the liquid at a pressure in a range between 500 and 1500 psi. Likewise, a preferred system of the invention may include the aforesaid pump(s).

The method of the invention may include providing the diluting liquid supply, and fluidly connecting the liquid supply to the diluter. Likewise, a preferred system of the invention may include the diluting liquid supply, fluidly connected to the diluter.

Preferably, the sanitising liquid is delivered to the one or more spraying and/or fogging devices by piping. A preferred method of the invention may including providing the piping. A preferred system of the invention may include said piping.

Preferably, the piping comprises one or more of, or any combination of, a copper, stainless steel, PVC or PEX piping. Preferably, the piping is approximately 25 mm in diameter.

The piping may comprise high pressure nylon tubing, for high pressure systems (e.g. fogging systems), or other piping or tubing known to the skilled addressee to be suitable for use in high pressure systems.

The features described in relation to one or more aspects of the invention are to be understood as applicable to other aspects of the invention. More generally, combinations of the steps in the method of the invention and/or the features of the system of the invention described elsewhere in this specification, including in the claims, are to be understood as falling within the scope of the disclosure of this specification.

The methods and/or systems of the invention may be applied as new sanitising systems or methods. However, the systems and/or methods of the invention are also suited to retrofit, or part retrofit, applications to existing equipment including delivery lines, spraying devices, pumps, diluters, controllers and/or control valves. The invention is conceived to take advantage of such equipment as such existing equipment may assist in delivering the sanitising solution to the locations contemplated by the invention.

Other aspects of the invention are also disclosed.

Persons skilled in the art will appreciate that many variations may be made to the invention without departing from the scope of the invention, which is determined from the broadest scope and claims.

### BRIEF DESCRIPTION OF THE DRAWINGS

Notwithstanding any other forms which may fall within the scope of the present invention, preferred embodiments of the invention will now be described, by way of example only, with reference to the accompanying drawings in which:

FIG. 1 is a schematic view of a first sanitising system in accordance with a preferred system of the invention;

FIG. 2 is a schematic view of a second sanitising system in accordance with a preferred system of the invention;

FIG. 3 is a flow diagram of the steps of a method for sanitising material including optional additional steps, in accordance with a preferred method of the invention;

FIG. 4 is a schematic view of a third sanitising system in accordance with a preferred system of the invention;

FIG. 5 is a schematic view of a fourth sanitising system in accordance with a preferred system of the invention;

### DETAILED DESCRIPTION

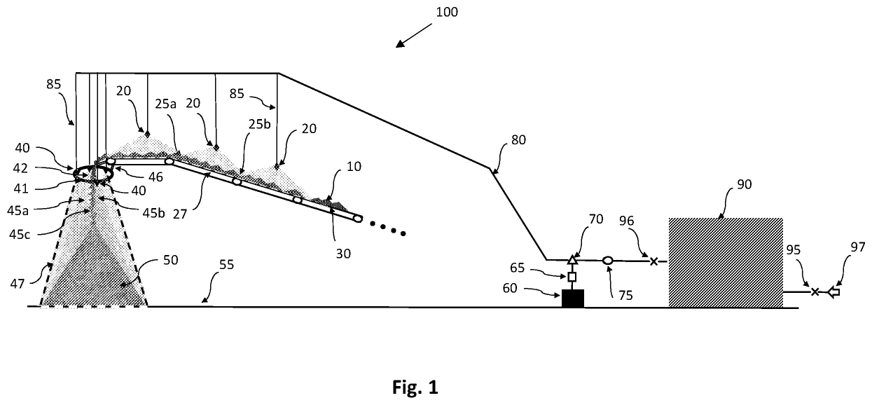

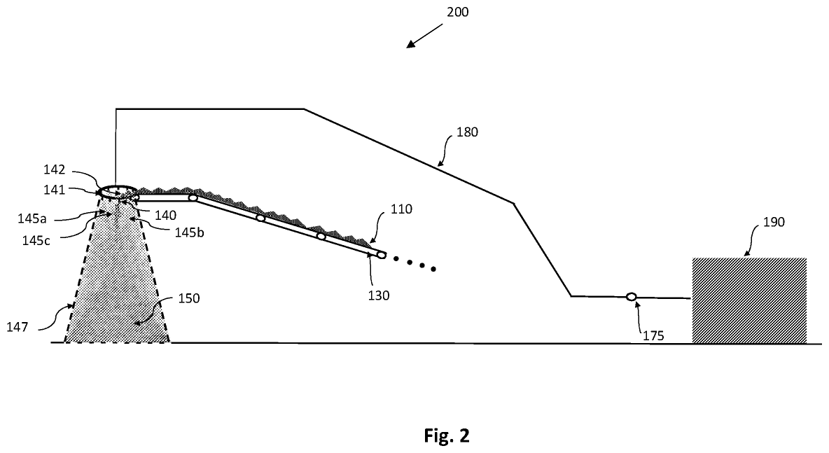

Referring to the FIGS. 1 and 2, there is depicted a first sanitising system 100 and a second sanitising system 200.

Referring to FIG. 1, the sanitising system 100 is for cleaning glass cullet material 10. The cullet comprises small segments that have a maximum thickness (diameter) of about 5 mm or less.

The cleaned culet (crushed recyclable glass material) can be used in many contexts, such as a sand substitute in concrete, or as a base or surface coat for roadways, or as an additive to clay for brick making, or to make fiberglass filaments for insulation, and in many other applications. Where the glass is pulverised and forms a fine material, in can be used in yet further applications, such as an underlay for walking trails, in dirt parking lots to keep dust down for filtration, and as a base for unpaved roads when mixed with soft clay soil.

The system 100 is configured to include seven spraying devices for spraying a sanitising liquid: three fan jets 20, and four cone spray jets 40 (only two of which are identified by reference numerals in FIG. 1). The three fan jets 20 spray the material 10 as it travels along a conveyor path 30, and the four cone spray jets 40 spray the material 10 as it exits and falls from the conveyor path 30. The four cone spray jets 40 also spray the material 10 as it forms a pile 50 below the conveyor path.

The three fan jets 20 provide two overlapping spray zones 25a, 25b in the conveyor path spray zone 27 (indicated by the dashed line on the conveyor path 30).

The four cone jets 40 are arranged to surround the material 10 as it exits the conveyor path 30, and the material 10 passes through a circular region 42 defined by the cone jets 40. The cone jets 40 are located on a ring 41 which defines the circular region 42. The ring 41, is attached to the conveyor belt by brackets 46 (only one of which is identified by reference numerals), so the ring 41 is located below the conveyor path 30. The diameter of the ring 41 is about 70 centimeters.

The cone jets 40 are arranged to produce a substantially conical spray zone 47 through which the material 10 passes as it exits the conveyor path 10. It will be appreciated that a greater proportion of spray is directed towards an inner region of, and towards the central vertical axis (not shown) of, the conical spray zone 47, which is broadly consistent with a higher proportion of the material being concentrated towards an inner region of the spray zone 47 and closer towards the central vertical axis thereof. The diameter of the conical spray zone 47 is about 7 meters at its base on the floor surface 55. The diameter of the conical spray zone 47 at its base extends to cover the pile 50 of material 10 forming below the conveyor path 30.

Within the conical spray zone 47 below the conveyor path 30 there are numerous overlapping spray zones (only some of which are identified in FIG. 1: 45a, 45b, 45c).

As a result of the conveyor belt passing around the roller at the end of the conveyor path 30, the change in direction of the small pieces of material as they pass over the end causes the material to rotate as it falls downwardly towards the floor 55 and pile 50. The cone jets 40 are adapted to spray the material 10 as it rotates while falling downwardly from the conveyor path 30. Additionally, the cone jets are arranged to spray the upper layer of material 10 as it forms on the pile 50 below the conveyor path 30. The spraying of the upper layer occurs for all of the time that the cone jets 40 are operating and there is pile 50, such that as the upper layer is constantly accreting by the addition of new material 10, so each newly accreted upper layer is coated with the sanitising liquid.

The seven liquid spraying devices 20, 40 are adapted to coat the entire, or substantially the entire, surface area of the glass cullet material 10 with the sanitising liquid. Where a surface area of the material 10 is not directly sprayed by the spraying devices 20, 40, the totality of the spraying (e.g. for a number of hours) including during the period where the material is forming in a pile 50, enables complete or substantially coverage of each of the small segments of material the sanitising liquid. The coverage of each of the small segments of material is assisted by sections of the pile which are frequently sliding and/or being displaced relative to other sections as the pile 50 accretes, ensuring different surfaces are being exposed to the liquid sprayed by the cone jets 40 over time. Additionally, relative movement of the small segments during the accreting of the pile 50 of material 10 can induce spread of the sanitising liquid where segments of the material 10 rub against each other.

The system 100 includes a liquid delivery line 80 that delivers the sanitising liquid to the seven spraying devices 20, 40, via seven separate spraying device lines 85 (only two of which are identified with reference numerals in FIG. 1). The delivery line 80 connects the spraying devices 20, 40 to a sanitising liquid supply.

The sanitising liquid is a composition comprising a mixture of a fatty acid:amino acid in a molar ratio between about 1:0.6 to about 1:1.6. More specifically, the composition comprises an aqueous solution including a mixture of the complex of decanoic acid:L-Arginine comprising between about 1% and 5% of the solution. As discussed later with reference to the diluting liquid supply, the diluted sanitising liquid has a lower the total percentage of active ingredients (e.g. where the sanitising liquid composition has 3% w/w active ingredients, and the composition is diluted 1:300 with water, then the total percentage of active ingredients in the diluted sanitising liquid is about 0.01%).

The composition is designed to be non-volatile and have no or negligible toxicity, for example as a food-safe fatty acid chain including capric acid. The composition may therefore be used in the context of food processing equipment as it is adapted to meet food-safe regulations such as those of the FDA.

In operation, once sprayed to the surface of a material having a cellular contaminant (e.g. a pathogen such as bacteria), the fatty acid acts to destabilize the lipid membrane. In particular, the fatty acid (e.g. capric acid) operates to break down the lipid bilayer cellular wall, which allows the complex of fatty acid and amino acid (e.g. fatty acid:Arginine) to enter the cell and inhibit pathogen growth. The complex of decanoic acid:L-Arginine as described above, is able to inhibit pathogen growth for long periods of time (i.e. for multiple days or multiple weeks, and for as long as 90 days and significantly longer if the concentration is increased, under certain conditions).

In some applications, the sanitising liquid provides sustained inhibition of pathogen growth for one or more weeks up to one or more months. In a particular arrangement, growth of bacteria on glass is inhibited for up to four months, or longer. In certain applications, sustained inhibition of pathogen growth may be provided at dilution ratios in a range of about 1:100 to 1:300 when diluted with water. Lower dilution ratios (i.e. providing increased amounts of the composition, per unit amount applied) provide a faster effective time to reduce the pathogen levels on the material to be sanitised.

A controlled amount of the sanitising liquid is dispensed into the diluted sanitising liquid line delivery 80, using a measuring unit 65 (e.g. a Bintech® or alternative liquid measuring device) drawing the liquid sanitising composition from a tote 60 containing about 1000 liters. The measuring unit 65 is connected to a diluter 70 (e.g. a venturi dilutor; MixRite® injector), and the diluter 70 is connected to the diluting liquid supply 90 so that the sanitising composition and diluting liquid can be diluted at a prescribed rate.

The diluter 70 produces a diluted sanitising liquid comprising a mixture of liquid sanitiser composition:diluting liquid in a ratio of about 1:300 (or about 1:200, or in between 1:200 and 1:300). Since the liquid sanitising composition is about 3% w/w active ingredients, the diluted sanitising liquid is between about 0.01% w/w (or up to about 0.015% w/w) of the total volume of diluted sanitising liquid sprayed by the spraying devices 20, 40. Where the liquid sanitiser composition is available at cost effective rates, lower dilution ratios (e.g. 1:50) may be applied.

The diluting liquid is water, stored in a tank 90 having a lid to prevent contamination of the water, and a capacity of about 40,000 liters. The tank 90 is connected to mains water 97, to continually replenish the water.

The diluting liquid is filtered using 5 micron filters 95, 96 in two locations near the inlet and outlet, respectively, of the diluting liquid supply 90. The filters 95, 96 remove solids from the diluting liquid and inhibit blockages of the spraying devices 20, 40, or the diluter 70/pump 75. The filters require changing every one or two weeks.

During the course of 8 hours, the sanitising system sprays between about 28,000 liters and 35,000 liters of diluted sanitising liquid, depending on the collective flow rate of the spraying devices.

The collective flow rate of the diluted sanitising liquid that is sprayed by the seven spraying devices 20, 40 in FIG. 1 is between about 1 to 1.2 liters per second. The spraying devices 20, 40 are configured to spray the sanitising liquid at approximately at the same rate through each the seven jets, so the collective flow through the four cone jets 40 is about 15% more of the overall flow compared to the flow through the three fan jets 20, per unit of time.

The diluting liquid is pumped by pump 75 from diluting fluid supply 90 to the diluter 70, and then to the spraying devices. The pump 75 is adapted pump the liquid at the desired flow rate (i.e. between 1 and 1.2 litres per second) at a pressure between about 40 PSI (or about 27 Newton per square centimetre) and about 120 PSI or (83 Newton per square centimetre).

The delivery line 80 comprises PEX piping approximately 25 mm in diameter.

The conveyor path 30 carries the material along the path at a rate of about 0.25 meters per second,

The depth of the material 10 on the conveyor path is between about 3 to about 5 centimeters at its highest. Of course, depending on the transfer of material to the conveyor path, there may be some variation in the material 10 height which does not reach the lower bound height of about 3 cm and/or exceeds the upper bound height of about 5 centimeters in some areas. The height of the material 10 on the conveyor is regulated to ensure that maximum coverage of the sanitising liquid is provided by the spraying devices.

The conveyor path 30, which is defined by a conveyor belt that circulates around rollers, is approximately 65 centimeters in width. The material 10 is spread across most of the width of that conveyor path 30.

The length of the conveyor path spray zone 27 (indicated by the dashed line) on the conveyor path 30 is approximately twenty (20) meters.

The end of conveyor path 30 is approximately 8 meters above the floor 55. Once the material reaches an end of the conveyor path 30, it exits the conveyor path and falls to the floor 55 in a pile 50. The pile 50 is continuously sprayed with diluted sanitising liquid by the four cone jets 40 as it accretes upwardly.

During the course of eight hours, the pile 50 starts to form and accretes to height of about 7 meters above the floor surface 55, two times. The base of the pile 50 has a diameter of about 7 meters across the floor 55, when the pile 50 height is about 7 meters.

The conveyor path 30 is adapted to carry about 1700 tonne of material 10 per 8 hours, so that the system sanitises two approximately 7-meter high piles of material 10 during that period of time.

With reference to FIG. 2, the sanitising system 200 is substantially the same as that described with reference to FIG. 1, except for the differences set out below.

The material 110 is hard recyclable plastic (e.g. PET or HDPE). There are no spraying devices for spraying on the conveyor path 130; the conveyor path 130 therefore does not include a conveyor path spray zone.

The one or more spraying devices comprise a spray bar 141 in the shape of a ring including ten (10) spray ring nozzles 140 (only one of which is identified on FIG. 2) adapted to deliver a conical spray zone 147. The ten nozzles provide numerous overlapping spray zones below the conveyor path 130 (only three of which are identified on FIG. 2: 145a, 145b, 145c). The spray ring 141 defines a region 142 through which the material 110 passes. The spray ring 141 is adapted to spray the material as it rotates and falls on the pile 150, including the upper layers of material 110 that accrete over time on the pile 150. The spray ring 141 is attached directly to the conveyor next to the rollers.

The spray ring 141 is adapted to coat the entire surface area of the plastic material with the sanitising liquid. Where a surface area of the material is not directly sprayed by the spray ring 141, the duration of spraying (e.g. for a number of hours) including during the period where the material is forming the pile 150, assists in providing complete, or substantially complete, coverage of the sanitising liquid for each of the small segments of material 110.

The sanitising liquid is pre-diluted then stored in a diluted sanitising liquid supply 190. The dilution ratio, the sanitising liquid and the diluent (water), remain the same as described with reference to FIG. 1. In this arrangement, therefore, there is no need for a diluter, measuring device, or separate concentrated sanitising liquid supply, as was provided in the system of FIG. 1. A pump 175 is provided to deliver the diluted sanitising liquid to the spray ring under the required pressure to enable spraying of the approximately 28,000 to 35,000 liters of diluted sanitising liquid over an 8 hour period. The collective flow rate of approximately 1 to 1.2 liters per second is delivered evenly by the ten spray ring nozzles 140.

There is only one line 180 delivering the diluted sanitising liquid to the spray ring 141 and each of the ten spray ring nozzles 140.

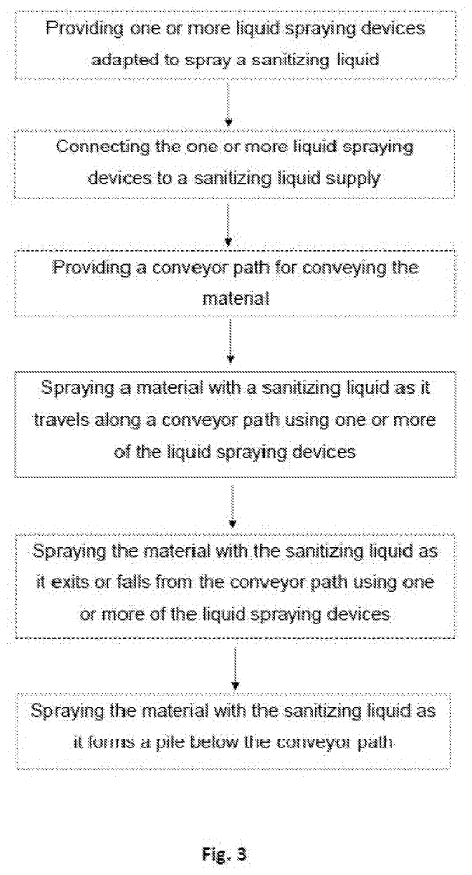

Referring to FIG. 3, there is shown a method of sanitising material, the method comprising: spraying a material with a sanitising liquid as it forms a pile below the conveyor path 360. The conveyor path, sanitising liquid and the liquid spraying devices are as described with reference to FIGS. 1 and 2.

Optional steps in the preferred method, indicated by the boxes with dashed lines, include: Providing the one or more liquid spraying devices adapted to spray a sanitising liquid 310; connecting the one or more liquid spraying devices to a sanitising liquid supply 320; providing a conveyor path for conveying the material 330; spraying the material with the sanitising liquid as it travels along a conveyor path using one or more liquid spraying devices 340; spraying the material with the sanitising liquid as it exits or falls from the conveyor path using one or more of the liquid spraying devices 350; Spraying can also provide dust suppression in applications where control of fine particles is required. The description corresponding to FIGS. 1 and 2 is also applicable to FIG. 3, in relation to the sanitising liquid, including the active ingredients and their proportions, the sanitising liquid supply, the pile, the flow rates, the nature and location of the spray zones, the type of spraying devices, the amount of material sanitised and other shared features of the system and method aspects of the invention.

Additionally, while not identified in FIG. 3, this preferred method of the invention may include any one or more of the following steps: providing the sanitising supply; providing a diluter and/or diluting the sanitising liquid with a diluting liquid; providing a measuring unit and/or dispensing a controlled amount of the sanitising liquid; and/or providing one or more filters and/or filtering the diluting liquid. The description corresponding to FIGS. 1 and 2 is also applicable to FIG. 3, in relation to the diluting step and/or the diluter, the controlled dispensing of the sanitising liquid step and/or measuring unit, and/or the filtering step and/or filters, pumps, etc.

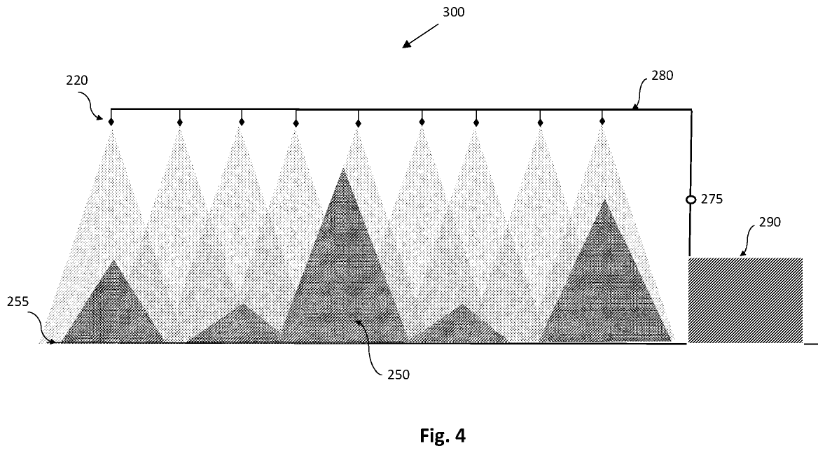

FIG. 4 presents a schematic view of a cleaning system 300 for a recyclable product storage facility that includes a plurality of misting sprayers 220 above piles of recycled product 250, piled on the storage facility floor 255. The sanitising liquid is pre-diluted and stored in a diluted sanitising liquid supply 290, then the pump 275 is adapted pump the liquid at the desired flow rate along the single line 280 that delivers to the nine misting sprayers 220.

While not described in FIGS. 1, 2 and 4, foaming and/or fogging application may replace and be preferable in many contexts to spraying liquid or misting sanitising liquid. Foaming and/or fogging reduces the amount of sanitising liquid that is needed to be applied as foaming, and particularly fogging, provides comparable results to liquid spraying using a proportionally reduced amount of the sanitising liquid. The foaming and/or fogging spraying devices can therefore be used with higher concentrations of the composition than those used for liquid spraying as described herein, to obtain the benefits of results using higher concentrations of the sanitising liquid.

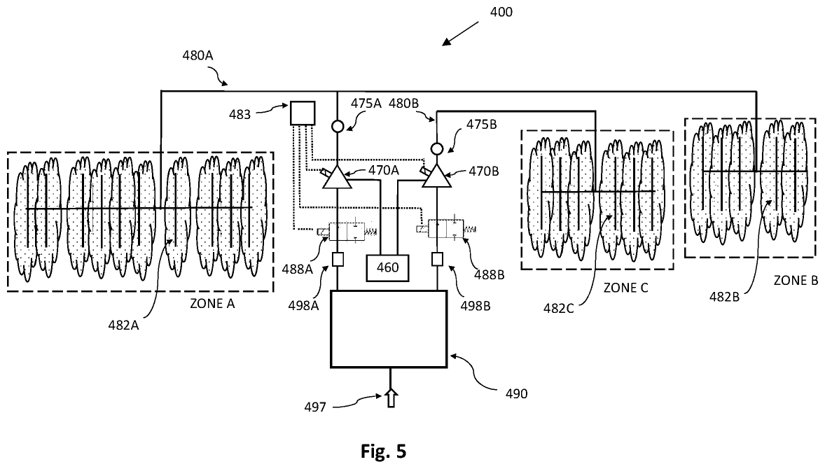

FIG. 5 illustrates an automatically controlled system 500 for multi-zoned fogging in a transfer station for recyclable material, including fine glass cullet. The system includes mains water 497 as a diluting liquid source connected to a diluting liquid supply (water tank) 490. The diluting liquid supply 490 is connected to two delivery lines 480A, 480B which themselves are connected to the fogging lines in each of the fogging zones A, B and C (labelled ZONE A, ZONE B, and ZONE C, respectively on FIG. 5, each being demarcated by a corresponding dashed line box).

In line along each of the sanitising liquid delivery lines 480A, 480B is, respectively, 5 micron filter 498A, 498B, one way solenoid valve 488A, 488B, controllable diluter 470A, 470B, and high pressure pump for increasing pressure in the diluted sanitising liquid in delivery lines 480A, 480B to approximately 1000 psi. Each of the controllable diluters 470A, 470B comprise an electronic metering pump connected to a sanitising liquid supply 460 comprising an 800 L enclosed container.

ZONE A, ZONE B and ZONE C each have a series of fogging lines 482A, 482B, 482C (only one of each fogging line in each ZONE is indicated by a reference numeral), each containing approximately 30 fogging nozzles (not shown) spaced 1 meter apart on the fogging line.

The diluters 470A, 470B and the solenoid valves 488A, 488B are controlled by PLC controller 483 so that a higher concentration of sanitising liquid is provided for ZONE C (1:100) compared to ZONE A and ZONE B (approx. 1:200). This is because ZONE C, which is an enclosed area, is for processing recyclable material that require a higher concentration of sanitising liquid to achieve optimal sanitising.

It will be appreciated that the timing and duration of delivery of sanitising liquid to the ZONES A and B can be controlled by PLC 483 independently from delivery of sanitising liquid to ZONE C, by the PLC independently opening and closing the solenoid valves 488A, 488B. Similarly, the rate of dilution of sanitising liquid in ZONES A and B can be adjustably controlled independently of the rate of dilution for ZONE C, in accordance with a preferred sanitising cycle (e.g. to raise and lower the concentration of sanitising liquid depending on the material to be sanitised, or the amount of pathogen control/elimination required in ZONES A and B, as compared to ZONE C, at any particular time.

In operation, since the recyclable material comprises glass cullet which is prone to being aerosolised as it is processed, fogging also acts as a dust suppressant to provide improved working conditions. Fogging also provides temperature control to reduce high temperature working conditions, and importantly, a benefit directly related to pathogen elimination is odour control (which minimises pest activity on the work site, or areas where processed and sanitised recyclable material is stored, where the sanitising is able to provide lasting benefits of many months).

Features of the system illustrated in FIG. 5 could similarly be applied in an automated system for sanitising a food processing facility. In such an application, foggers may be localised to sanitise drains, conveyors, bins, benches, etc. and also air filters for pushing food along tables. Such a system would be automated and centrally controlled by a PLC. In further detail, the set-up would include dosing drains at regular intervals during the course of a day. Further, the first sanitising operation of each month may be conducted at a higher concentration of sanitising liquid:diulent (1:10), to ensure hard surfaces receive an adequate coating to provide long term protection. Further, everyday fogging sanitising operation, the system may be programmed to dilute the sanitising liquid at rate of 1:50. The system would be automated for continuous application or at regular intervals during processing, to food carrying conveyors, bins, etc and into the floor drains to assist in bacterial elimination.

It will be understood that the systems and methods of the invention are not limited to providing sanitising only, and may be applied in a variety of contexts including waste processing and recycling industries (e.g. resource recovery centres, transfer stations and other solid waste management centres), manufacturing and agricultural settings, food processing/storage and food packaging. The methods and systems of the invention are adapted to provide, in parallel with sanitising, means to control or improve working conditions or a site more generally, such as control of dust, odour and/or pests. As such, benefits of the invention are manifold.

In indoor settings in particular, it is often recommended that people do not enter rooms being sanitised where sanitiser is applied as a fog, mist or vapour (or where the sanitiser has any toxicity), as concentrations may cause eye/skin irritation or damage if people enter an area undergoing treatment. In the context of food processing or food preparations areas, past practices have involved spraying a foaming agent, scrubbing areas to be cleaned, waiting for 10 minutes, rinsing the areas, clearing staff from the work area, cleaners dressed in high level PPE applying areas with a toxic cleaning agent (e.g. Peracetic Acid, such as Proxitane®), leaving the areas for 15 minutes, then rinsing down the areas before staff are allowed to return. It will be understood that since the method and system of the system involves application of a food-safe sanitising liquid, there is no health risk to the workers so that the sanitising liquid can be applied (e.g. fogged, foamed or sprayed) without requiring workers to leave the work area.

It will be understood that fogging is particularly preferred in indoor settings, where this form of spraying is less impacted by wind or other weather conditions. Fogging is particularly effective in food preparation or food processing areas where a high concentration of sanitising liquid is preferred due to the need for rapid and consistent removal of pathogens (and in particular, ensuring food that is to be is packed is effectively sanitised). In such environments, the benefits of fogging are many, as this mode of application allows for a higher dilution rate due to lower flow rates thereby providing equally effective, or better, sanitising than equivalent spraying, misting or foaming application, per unit of sanitising liquid used. The method of fogging also provides a deeper penetration of the sanitising liquid into the surface (or the produce) which enables longer lasting sanitisation.

Other benefits provided by the invention include:

Harmful pathogens are eliminated from materials that are treated and/or equipment or machinery, thereby creating a safer working environment for the staff;

The elimination of pathogens such as bacteria, to prevent malodour at an early, or the first practical stage, works better than alternative methods that try and conceal the odour or enclose the material which are generally ineffective at preventing pathogen growth;

The prevention of malodour will contribute to a reduction in rodent and bird activity at the location;

The sanitising liquid can be derived from naturally occurring food safe ingredients, and is environmentally friendly;

The wetting agent properties of the sanitising liquid make it a very effective dust suppressant;

Application of the sanitising liquid in accordance with the methods and systems of the invention limits pathogen growth as the sanitising liquid remains on the surfaces to it binding to the pathogens at a molecular level and achieving long lasting sanitising;

The sanitising liquid is non-corrosive so it will not degrade the dispensing equipment in the system as much as other sanitising liquids (e.g. limiting the occurrence of blockages in the nozzles and jets);

The sanitising liquid is adapted to extend the lifespan of agricultural produce or other food, once it is sprayed thereon;

The sanitising liquid works well over the long term, and operates to control odours are caused by pathogens (and thereby also reduces rodents or other pest activity since they are not drawn to the material by the smell);

It operates to reduce or eliminate pathogens on materials or surfaces, and where it is applied at the appropriate concentration and in the appropriate method of spraying application, the sanitising liquid spreads across and coats the surface of the material and thereby deliver long term protection against pathogens;

The systems and methods of the invention eliminate or inhibit pathogen growth and thereby remove malodours and the dangers of pathogenic bacteria (e.g. where it leaches from roads into water ways or negatively impacts laborers using the materials).

### INDUSTRIAL APPLICABILITY

It is apparent from the above, that the arrangements described are applicable to industries, such as industrial recycling plants and food processing or packaging facilities, in which the sanitisation of material and surfaces has commercial and practical implications.

### GENERAL STATEMENTS

### Embodiments

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that each unit between two particular units are also disclosed. For example, if 10 and 15 are disclosed, then 11, 12, 13, and 14 are also disclosed.

### Different Instances of Objects

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

### Specific Details

In the description provided herein, numerous specific details are set forth. However, it is understood that embodiments of the invention may be practiced without these specific details. In other instances, well-known methods, structures and techniques have not been shown in detail in order not to obscure an understanding of this description.

### Terminology

The terms in the claims have the broadest scope of meaning they would have been given by a person of ordinary skill in the art as of the relevant date.

The term “material” should be understood as broadly encompassing and include aggregate material (e.g. crushed recyclable material), produce (e.g. fresh produce, agricultural produce), food producing plants, hard surfaces (e.g. floors), and other items as referred to in the specification.

The term “spray” and variations such as “spraying” or “sprayer” should be understood to include a range of methods and devices for propelling or driving water, usually in droplet or particle form. The term “sprayer” or “spraying device” is intended to capture devices such as spray guns, high pressure jet sprayers, conventional pressure sprayers, pressure washers, misting devices, foaming devices, fogging devices, vaporisers and atomisers, and the term “spray” or “spraying” refers to applications of such devices.

The terms “a” and “an” mean “one or more”, unless expressly specified otherwise

Neither the title nor any abstract of the present application should be taken as limiting in any way the scope of the claimed invention.

Where the preamble of a claim recites a purpose, benefit or possible use of the claimed invention, it does not limit the claimed invention to having only that purpose, benefit or possible use.

In the present specification, terms such as “part”, “component”, “means”, “section”, or “segment” may refer to singular or plural items and are terms intended to refer to a set of properties, functions or characteristics performed by one or more items having one or more parts. It is envisaged that where a “part”, “component”, “means”, “section”, “segment”, or similar term is described as consisting of a single item, then a functionally equivalent object consisting of multiple items is considered to fall within the scope of the term; and similarly, where a “part”, “component”, “means”, “section”, “segment”, or similar term is described as consisting of multiple items, a functionally equivalent object consisting of a single item is considered to fall within the scope of the term. The intended interpretation of such terms described in this paragraph should apply unless the contrary is expressly stated or the context requires otherwise.

The term “connected” or a similar term, should not be interpreted as being limitative to direct connections only. Thus, the scope of the expression an item A connected to an item B should not be limited to items or systems wherein an output of item A is directly connected to an input of item B. It means that there exists a path between an output of A and an input of B which may be a path including other items or means. “Connected”, or a similar term, may mean that two or more elements are either in direct physical or electrical contact, or that two or more elements are not in direct contact with each other yet still co-operate or interact with each other.

### Comprising and Including

In the claims which follow and in the preceding description of the invention, except where the context requires otherwise due to express language or necessary implication, the word “comprise” or variations such as “comprises” or “comprising” are used in an inclusive sense, i.e. to specify the presence of the stated features but not to preclude the presence or addition of further features in various embodiments of the invention.

Any one of the terms: including or which includes or that includes as used herein is also an open term that also means including at least the elements/features that follow the term, but not excluding others. Thus, including is synonymous with and means comprising.

### Scope of Invention

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used.

Functionality may be added or deleted from the block diagrams and operations may be interchanged among functional blocks. Steps may be added or deleted to methods describe within the scope of the present invention.

Although the invention has been described with reference to specific examples, it will be appreciated by those skilled in the art that the invention may be embodied in many other forms.

1.-7. (canceled)

8. A method of sanitising an aggregate material, the method including the steps of:
providing one or more sanitising liquid delivery lines that receive a sanitising liquid;
connecting the one or more delivery lines to one or more liquid spraying devices;
applying the sanitising liquid to the aggregate material, using the one or more liquid spraying devices, including by one or the following process(es):
spraying the materials as it travels along a conveyor path;
spraying the material as it exists or falls from a conveyor;
spraying a pile of the material;
wherein
the sanitising liquid comprises a composition including decanoic acid in a range between about 1% and about 5% w/w; and
the sanitising liquid composition is diluted with a diluent.

9. An aggregate material sanitising system, the system comprising:
a sanitising liquid delivery line for receiving a sanitising liquid;
one or more liquid spraying devices connected to the delivery line;
wherein
the one or more liquid spraying devices are configured to apply sanitizing liquid to the aggregate material, including by one or more of the following process(es):
spraying the material as it travels along a conveyor path;
spraying the material as it exits or falls from a conveyor;
spraying a pile of the material;
the sanitising liquid comprises a composition including decanoic acid in a range between about 1% and about 5% w/w; and
the sanitising liquid composition is diluted with a diluent.

10. The method according to claim 8, wherein the aggregate material is a recyclable material.

11. The method according to claim 10, wherein the recyclable material is crushed glass, pulverised glass, or glass cullet.

12. The method according to claim 8, wherein the one or more liquid spraying devices comprise one or more fogging devices and/or misting devices adapted to spray the material in an indoor environment.

13. The method according to claim 12, wherein the sanitising liquid is diluted to a concentration in a range between about 1:150 and about 1:250 sanitising liquid composition:diluent for application in the indoor environment by the one or more fogging devices.

14. The method of system according to claim 8, wherein the material comprises dust particles and the one or more liquid spraying devices comprise fogging devices adapted to spray a fog to suppress the dust particles.

15. The method according to claim 8, wherein the one or more liquid spraying devices comprise misting devices and/or conventional spraying devices adapted to spray the material in an outdoor environment.

16. The method according to claim 8, wherein the one or more liquid spraying devices comprise one or more spray rings.

17. The method according to claim 16, wherein the material passes through one or more spray rings as it exits or falls from the conveyor.

18. The method according to claim 8, wherein the method includes spraying the material with the sanitising liquid as it forms the pile, and the pile is below the conveyor path.

19. The method according to claim 8, wherein application of the sanitising liquid provides effective sanitising for at least 30 days.

20. (canceled)

21. (canceled)

22. (canceled)

23. The method according to claim 8, wherein the method includes the step of providing, or the system comprises, a controller adapted to control flow of the sanitising liquid along the delivery line and to the one or more liquid spraying devices.

24. The method according to claim 23, wherein the controller is adapted to provide zoned and/or timed application of the sanitising liquid.

25. The method according to claim 8, wherein the sanitising liquid is a composition including a mixture of the decanoic acid and an amino acid.

26. The method according to claim 25, wherein the amino acid is L-Arginine.

27. The method according to claim 8, wherein the method includes the step of providing, or the system comprises, a diluter for diluting the sanitising liquid with the diluent.

28. (canceled)

29. The method according to claim 8 wherein the sanitizing liquid composition is diluted to a concentration in a range between about 1:100 and about 1:300 sanitising liquid composition:dilutent.

30. The method according to claim 8, wherein the aggregate material is organic or partly organic material.

31. The method according to claim 30, wherein the aggregated material is soil.
